(19) United States
(12) Patent Application Publication
(10) Pub. No.: US 2019/0046007 A1
(43) Pub. Date: Feb. 14, 2019

(54) ENDOSCOPIC VIDEO RECORDING SYSTEM WITH VOICE CONTROL AND VOICE ANNOTATION

(71) Applicant: Virgo Surgical Video Solutions, Inc., San Francisco, CA (US)

(72) Inventors: Matthew Zane Schwartz, San Francisco, CA (US); David Oscar Guaraglia, San Francisco, CA (US); Ian Christopher Strug, Chicago, IL (US)

(21) Appl. No.: 15/929,035

(22) Filed: Aug. 2, 2018

Related U.S. Application Data
(63) Continuation-in-part of application No. 15/793,502, filed on Oct. 25, 2017.
(60) Provisional application No. 62/540,371, filed on Aug. 2, 2017.

(51) Int. Cl. A61B 1/00; A61B 1/04; G06F 3/16

(52) U.S. Cl. CPC A61B 1/00041; A61B 1/04; G06F 17/241; A61B 1/00043; G06F 3/167

(57) ABSTRACT

An endoscopic video recording system with voice control and voice annotation. By utilizing voice control, the system enables hands-free setup and control of video recording. The system is maintained in a “ready for use” state, so clinicians can activate it at any time using voice control.

### CROSS-REFERENCE TO RELATED APPLICATIONS

This application is a continuation-in-part of application Ser. No. 15/793,502, filed on Oc. 25, 2017, and claims the benefit of priority under 35 § 119(e) to U.S. Provisional Patent Application No. 62/540,371, entitled “An endoscopic video recording system with voice control and voice annotation,” filed on Aug. 2, 2017. Each of these applications is hereby incorporated by reference herein in its entirety.

### FIELD OF THE INVENTION

This disclosure pertains to systems and methods for video recording in an endoscopy system, and more particularly, to systems and methods for providing physicians' annotations to a video recording produced for an endoscopy system.

### BACKGROUND

Medical practitioners often wish to record clinical video of their diagnostic and surgical procedures. However, they rarely have the time or knowledge to setup video recording systems. A user friendly video recording system does not exist for these physicians.

Before, during, and after medical procedures, clinicians do not have the time or desire to manually setup video recording systems. Existing systems require manual setup for video recording.

By utilizing voice control, this system enables hands-free setup and control of video recording. This is a much simpler and more user-friendly system than existing recording systems. This system is always ready for use, so clinicians can activate it at any time using voice control.

It would also be advantageous to utilize voice control in providing voice annotations to video recording. Systems that provide voice and other audio annotations to video and image data are known. For example, U.S. Pat. No. 6,721,001 to Berstis (“Digital Camera With Voice Recognition Annotation”), which issued Apr. 13, 2004 and is hereby incorporated by reference in its entirety herein, discloses a digital camera that allows voice annotations to be recorded with each digital picture rendered by the camera. When transmitting a digital picture from the camera to another device, the camera transforms the associated voice annotation to a text annotation.

U.S. Pat. No. 7,324,943 to Rigazio et al. (“Voice Tagging, Voice Annotation, and Speech Recognition for Portable Devices with Optional Post Processing”), which issued Jan. 29, 2008 and is hereby incorporated by reference in its entirety herein, discloses a media annotator that is operable to recognize an audio input made close in time to the capture of a media segment, and to annotate the media segment with a text sample prepared from the captured audio input by a speech recognizer.

U.S. Pat. No. 8,385,588 to Jacoby et al., which issued on Feb. 26, 2013 and is hereby incorporated by reference in its entirety herein, discloses a method for processing audio signals representing commentary on an associated media file for representation as text-based metadata associated with the media file.

### SUMMARY

By way of example, aspects of the present disclosure are directed to methods for providing an endoscopic video recording system with voice control and voice annotation. By utilizing voice control, the system enables hands-free setup and control of video recording. The system is maintained in a “ready for use” state, so clinicians can activate it at any time using voice control.

In particular, a computer-implemented method is disclosed for voice annotation of a video recording produced by video recording system of an endoscopy system, includes the steps of:

a. detecting that the video recording system is actively recording video content or in a video recording active ready mode;

b. receiving an indication that a voice command to begin recording was received via an audio input mechanism;

c. recording voice annotation content based on a voice annotation signal received by the audio input mechanism;

d. terminating the recording step upon receiving an indication that a voice command to stop recording was received via the audio input mechanism; and

e. storing the recorded video content and the recorded voice annotation content for retrieval via a front-end user interface.

In accordance with another aspect of the disclosure, the recording step of the computer-implemented method may include the step of converting voice data in the voice annotation content to text note data.

In accordance with another aspect of the disclosure, the computer-implemented method may include a step for tagging at least a portion of the voice annotation content with flag or other indicator upon receiving an indication that an associated voice command was received by the audio input mechanism. The indicator may, for example, be an importance indicator.

In accordance with another aspect of the present disclosure, the voice command may instruct the recording of a lengthened note with a playing time that exceeds that playing time of the associated recorded video content.

In accordance with yet another aspect of the present disclosure, the front-end user interface may be provided as an integrated component of the endoscopy system. Alternatively, the front-end user interface may be provided as an adjunct computer.

This SUMMARY is provided to briefly identify some aspects of the present disclosure that are further described below in the DESCRIPTION. This SUMMARY is not intended to identify key or essential features of the present disclosure nor is it intended to limit the scope of any claims.

### BRIEF DESCRIPTION OF THE DRAWINGS

A more complete understanding of the present disclosure may be realized by reference to the accompanying drawings in which:

FIG. 1 provides a schematic view of an endoscopic video recording system according to principles of the present disclosure a; and

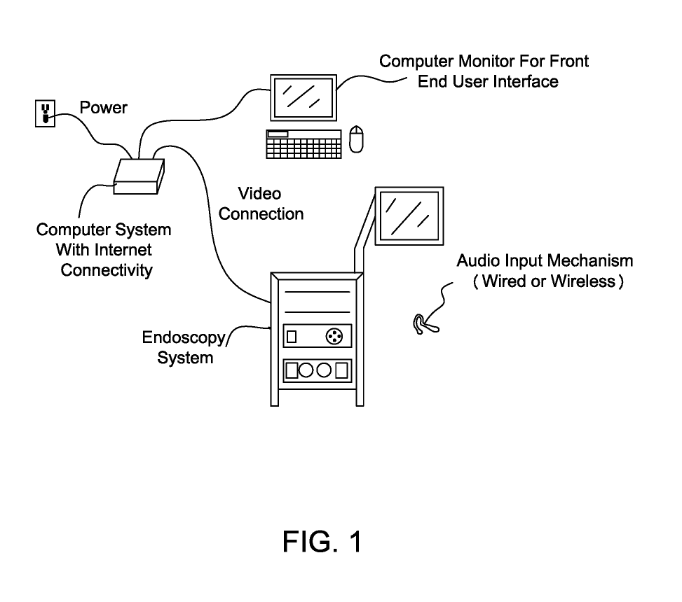

FIG. 2 illustrates a method for operating the endoscopic video recording system of FIG. 1.

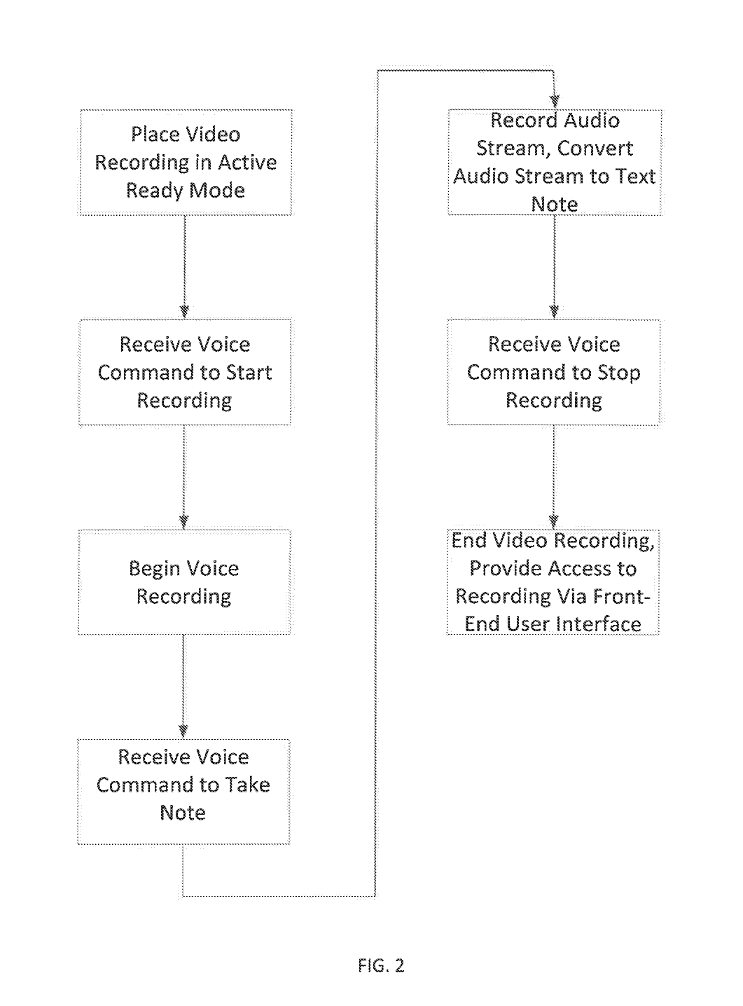

### DETAILED DESCRIPTION

The following merely illustrates the principles of the disclosure. It will thus be appreciated that those skilled in the art will be able to devise various arrangements which, although not explicitly described or shown herein, embody the principles of the disclosure and are included within its spirit and scope.

Furthermore, all examples and conditional language recited herein are principally intended expressly to be only for pedagogical purposes to aid the reader in understanding the principles of the disclosure and the concepts contributed by the inventor(s) to furthering the art, and are to be construed as being without limitation to such specifically recited examples and conditions.

Moreover, all statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements later developed that perform the same function, regardless of structure.

Unless otherwise explicitly specified herein, the drawings are not drawn to scale.

In the following description, the same reference signs are used for the same and similarly acting parts.

As stated above, medical practitioners often wish to record clinical video of their diagnostic and surgical procedures. However, they rarely have the time or knowledge to setup video recording systems. A user friendly video recording system does not exist for these physicians. The invention claimed here solves this problem.

By utilizing voice control and voice annotation, this system facilitates simplified medical video recording. Voice control enables hands-free start and stop of video recording. Voice annotation allows physicians to denote specific sections of the video recording as important and take notes.

The methods and systems disclosed herein differ from what currently exists. Existing video recording systems require manual input such as pushing physical buttons or touch screens to format the video recording and to start and stop the recording.

The methods and systems disclosed herein improve on what currently exists. Existing video recording systems require manual input such as pushing physical buttons or touch screens to format the video recording and to start and stop the recording.

Existing medical video recording system are hard to operate. Clinicians rarely know how to use them, and when they do know, they don't have the time to use them for medical procedures.

By utilizing voice control, the methods and systems disclosed herein enable hands-free setup and control of video recording. This is a much simpler and more user-friendly system than existing recording systems. This system is always ready for use, so clinicians can activate it at any time using voice control.

Also, the methods and systems disclosed herein can produce other useful items such as audio and video recordings of medical procedures, as well as medical annotations and notes which may be used for medical records.

In accordance with aspects of the present disclosure, a system for voice control and voice annotation of diagnostic and surgical procedures may preferably include: 1) A computer with audio and video input capabilities, 2) video content, 3) audio content, 4) an audio recording device, 5) a video player allowing for direct digital video recording, 6) an audio player allowing for direct digital audio recording, 7) optional analog to digital converters for audio and video, 8) a computer monitor, 9) a mouse or other manually manipulatable interface for controlling on-screen cursor activity, 10) a computer keyboard, 11) software controlling the audio and video inputs, 12) software converting the audio and video streams into useful data for recording control and annotation, 13) software that creates the user interface for interacting with video, audio, and text, 14) hardware for internet connectivity, and 15) cloud computing and storage services.

All computer peripherals (audio recording device (4), video player allowing direct digital video recording (5), audio player allowing direct digital audio recording (6), optional analog to digital converters (7), computer monitor (8), mouse or other manually manipulatable input (9), and computer keyboard (10) along with any required hardware) are connected to the computer with audio and video capabilities (1). Software controlling audio and video input (11), software converting audio and video input to data for recording control and annotation (12), and software that creates the user interface is installed on the computer with audio and video capabilities (1) or other local or cloud based computer systems to provide access to users. Hardware for internet connectivity (14) is connected or installed to computer with audio and video capabilities (1), such that video content (2), audio content (3), converted text data, and other useful data or metadata may be stored locally or stored using cloud computing and storage services (15).

The overall function of the system is to simplify endoscopic video recording and enable new functionality such as voice based annotation. Voice controlled commands such as “start recording” and “stop recording” enable always active, hands free recording. Voice based annotation allows users to annotate their video or audio content in a hands free manner. The software of the system, between data acquisition, data parsing, back end services, and front end services ensures that data flows properly throughout the system and proper recordings are created.

Once setup is complete and the software has been properly installed, the system will be ready to receive audio and video input from a physician and endoscopy system respectively. During an endoscopic procedure the video recording system will be active. The computer will receive audio input from the physician including commands such as “start”, “stop”, “record”, “flag”, “important”, “make note”, etc. These inputs will trigger actions of the recording system accordingly. For example, if the physician commands “start recording”, then the system will start recording audio and/or video. In situations where the physician issues a command such as “make note”, they will be able to then speak a longer note into the system for recording and annotation. Following the recording of audio and video content, the back end system software will organize data and associated metadata. This data and metadata will then be appropriately made available to physicians and other end users.

The user of the invention first sets up or installs the hardware system in conjunction with an endoscopy system. This typically involves connecting the video output of the endoscopy system to the video input of the recording system. The invention should also be connected to the appropriate power source and internet connection. Once setup, the user can begin performing endoscopic procedures and using the provided audio input mechanism to control various aspects of the recording. The user may also use the audio input mechanism to provide annotation to the video or annotation to be used without the recorded video.

It will be understood that, while various aspects of the present disclosure have been illustrated and described by way of example, the invention claimed herein is not limited thereto, but may be otherwise variously embodied within the scope of the following claims. For example, various configurations of audio, video and other data inputs may be used. Specifically, it could be useful to include multiple audio or video inputs. Additionally, the various inputs, computing systems, and software systems may be spread across multiple devices, rather than being housed within a single device. Additionally, while the disclosed system is intended to be used in the field of medicine (specifically endoscopy), it is possible that the system may be used to improve video recording in other fields such as manufacturing and other services.

We claim:

1. A computer-implemented method for voice annotation of a video recording produced by video recording system of an endoscopy system, the method comprising the steps of:
a. detecting that the video recording system is actively recording video content or in a video recording active ready mode;
b. receiving an indication that a voice command to begin recording was received via an audio input mechanism;
c. recording voice annotation content based on a voice annotation signal received by the audio input mechanism;
d. terminating the recording step upon receiving an indication that a voice command to stop recording was received via the audio input mechanism; and
e. storing the recorded video content and the recorded voice annotation content for retrieval via a front-end user interface.

2. The computer-implemented method of claim 1, wherein the step of recording voice annotation content further comprises the step of converting voice data in the voice annotation content to text note data.

3. The computer-implemented method of claim 1, further comprising the step of tagging at least a portion of the voice annotation content with flag or other indicator upon receiving an indication that an associated voice command was received by the audio input mechanism.

4. The computer-implemented method of claim 3, wherein the flag comprises an importance indicator.

5. The computer-implemented method of claim 1, wherein the associated voice command instructs the recording of a lengthened note based on the voice annotation content that may exceed the recorded video content in length.

6. The computer-implemented method of claim 5, wherein a playing time of the voice annotation content for the lengthened note exceeds a playing time for the associated recorded video content.

7. The computer-implemented method of claim 1, wherein the front-end user interface is provided as an integrated component of the endoscopy system.

8. The computer-implemented method of claim 1, the front-end user interface is provided as an adjunct computer.
